(19) United States
(12) Patent Application Publication
(10) Pub. No.: US 2025/0201375 A1
(43) Pub. Date: Jun. 19, 2025

(54) Leveraging Large Language Models for Automating Lines of Therapy Adjudication in Cancer Patients

(71) Applicant: Bristol-Myers Squibb Company, Princeton, NJ (US)

(72) Inventors: Shamsulhaq Basir, Princeton, NJ (US); Md Shamsuzzaman, Lawrenceville, NJ (US)

(73) Assignee: Bristol-Myers Squibb Company, Princeton, NJ (US)

(21) Appl. No.: 18/986,183

(22) Filed: Dec. 18, 2024

Related U.S. Application Data
(60) Provisional application No. 63/611,977, filed on Dec. 19, 2023.

(51) Int. Cl. G16H 20/10; G06F 16/3329; G16H 10/60; G16H 15/00

(52) U.S. Cl. CPC G16H 20/10; G06F 16/33295; G16H 10/60; G16H 15/00

(57) ABSTRACT

A method includes receiving natural language input text characterizing clinical data for a patient. The method also includes receiving a prompt composition that includes adjudication rules for performing lines of therapy adjudication and an instruction parameter that specifies a task for a LLM to synthesize a group of multiple synthetic experts that each use the adjudication rules to perform chain-of-thought reasoning for making lines of therapy (LoT) adjudication decisions. The method also includes structuring an adjudication prompt by concatenating the prompt composition to the natural language input text, processing, using the LLM, the adjudication prompt to cause the LLM to synthesize the group of multiple synthetic experts and generate a respective group answer. The method also includes determining a final answer based on the respective group answer generated from the group of multiple synthetic experts.

### CROSS-REFERENCE TO RELATED APPLICATIONS

This U.S. patent application claims priority under 35 U.S.C. § 119(e) to U.S. Provisional Application 63/611,977, filed on Dec. 19, 2023. The disclosure of this prior application is considered part of the disclosure of this application and is hereby incorporated by reference in its entirety.

### TECHNICAL FIELD

This disclosure relates to leveraging large language models (LLMs) for automating lines of therapy adjudication in cancer patients.

### BACKGROUND

Lines of therapy (LoT) is refers to the order of different treatment regimens given to a patient diagnosed with a particular disease as their disease progresses. The number of prior LoTs influence the clinical benefit and even the magnitude of response to select treatment regimens for treating the particular disease. Prior LoT is crucial for optimizing treatment, medication audits, and clinical trial eligibility.

### SUMMARY

One aspect of the disclosure provides a computer-implemented method that when executed on data processing hardware causes the data processing hardware to perform operations for performing lines of therapy (LoT) adjudication automatically by leveraging a large language model (LLM). The operations include receiving natural language input text characterizing clinical data for a patient diagnosed with a particular disease. The clinical data includes: patient information including a name of the particular disease the patient is diagnosed with and an initial diagnosis date of the particular disease; treatment information including a number of different treatment regimens administered to the patient for treating the particular disease; and outcome information indicating treatment responses to the treatment regimens administered to the patient. The operations also include receiving a prompt composition. The prompt composition includes adjudication rules that specify rules for performing lines of therapy adjudication and an instruction parameter that specifies a task for a large language model (LLM) to synthesize a group of multiple synthetic experts that each use the adjudication rules to perform chain-of-thought reasoning for making lines of therapy (LoT) adjudication decisions based on the natural language input text and collaborate with one another to agree upon a predicted number of LoT administered to the patient. The operations also include structuring an adjudication prompt by concatenating the prompt composition to the natural language input text and processing, using the LLM, the adjudication prompt to cause the LLM to synthesize the group of multiple synthetic experts and generate a respective group answer as output from the group of multiple synthetic experts. The group answer indicates the predicted number of LoT agreed upon by each synthetic expert in the group of multiple synthetic experts. The operations also include determining a final answer based on the respective group answer generated as output from the group of multiple synthetic experts. The final answer includes an adjudicated list of LoT for each of the number of different treatment regimens administered to the patient.

Implementations of the disclosure may include one or more of the following optional features. In some implementations, each corresponding treatment regimen of the number of different treatment regiments administered to the patient is paired with a description of the corresponding treatment regimen, a start date indicating when the patient began the corresponding treatment regimen, and an end date indicating when the patient stopped the corresponding treatment regimen. In some additional implementations, the prompt composition further includes a format parameter that specifies how the LLM should format the respective group answer generated as output from the group of multiple synthetic experts. Here, the format of the respective group answer specified by the format parameter may include a list of elements containing numerical values of LoT for each treatment regimen of the number of different treatment regimens administered to the patient, wherein the format parameter specifies that a number of elements in the list of elements must be equal to the number of different treatment regimens.

In some examples, the prompt composition further includes one or more few-shot learning examples that each include exemplary natural language text characterizing example clinical data for an example patient being treated for the particular disease and a corresponding ground-truth list of LoT for each treatment regimen of the multiple different treatment regimens administered to the example patient. The example clinical data characterized by the natural language text includes multiple different treatment regimens administered to the example patient. In these examples, each few-shot learning example may further include a corresponding ground-truth chain-of-thought reasoning for why an expert would adjudicate the corresponding ground-truth list of LoT from the exemplary natural language text. Optionally, the instruction parameter that specifies the task for the LLM to synthesize the group of multiple synthetic experts that each use the adjudication rules to perform the chain-of-thought reasoning for making the LoT adjudication decisions may further specify that each synthetic expert is to also use the one or more few-shot learning examples to perform the chain-of-though reasoning for making the LoT adjudication decisions.

In some implementations, at least one of the different treatment regimens administered to the patient includes a combination of one or more drugs. Here, the prompt composition may further include a mechanism of action parameter that specifies: one or more drug classes commonly used to treat the particular disease; and for each corresponding drug class of the one or more drug classes, an example list of drugs within the corresponding drug class that have a same mechanism of action. Receiving the natural language input text may include receiving a clinical data table for the patient and serializing the clinical data table into the natural language input text. The clinical data table stores the clinical data for the patient in a tabular form.

In some examples, processing the adjudication prompt to cause the LLM to synthesize the group of synthetic experts includes, for each corresponding LLM instance of multiple LLM instances of the LLM, instructing the corresponding LLM instance to process the adjudication prompt independently from the other LLM instances to cause the corresponding LLM instance to synthesize a respective group of synthetic experts and generate a respective group answer as output from the respective group of multiple synthetic experts that indicates the predicted number of LoT agreed upon by each synthetic expert in the respective group of multiple synthetic experts. Here, the LLM may include a single pre-trained LLM executing each of the multiple LLM instances or the LLM may include two or more different pre-trained LLMs each executing one or more respective LLM instances among the multiple LLM instances. In these examples, the operations may further include determining a majority answer among the respective group answers generated as output from the respective groups of multiple synthetic experts synthesized by the multiple LLM instances of the LLM, wherein determining the final answer may include determining the final answer as the determined majority answer. Additionally, the operations may further include determining a majority vote percentage based on a total number of groups and a number of the respective groups of multiple synthetic experts that generated the same respective group answer as the majority answer.

Another aspect of the disclosure provides a system that includes data processing hardware and memory hardware storing instructions that when executed on the data processing hardware causes the data processing hardware to perform operations. The operations include receiving natural language input text characterizing clinical data for a patient diagnosed with a particular disease. The clinical data includes: patient information including a name of the particular disease the patient is diagnosed with and an initial diagnosis date of the particular disease; treatment information including a number of different treatment regimens administered to the patient for treating the particular disease; and outcome information indicating treatment responses to the treatment regimens administered to the patient. The operations also include receiving a prompt composition. The prompt composition includes adjudication rules that specify rules for performing lines of therapy adjudication and an instruction parameter that specifies a task for a large language model (LLM) to synthesize a group of multiple synthetic experts that each use the adjudication rules to perform chain-of-thought reasoning for making lines of therapy (LoT) adjudication decisions based on the natural language input text and collaborate with one another to agree upon a predicted number of LoT administered to the patient. The operations also include structuring an adjudication prompt by concatenating the prompt composition to the natural language input text and processing, using the LLM, the adjudication prompt to cause the LLM to synthesize the group of multiple synthetic experts and generate a respective group answer as output from the group of multiple synthetic experts. The group answer indicates the predicted number of LoT agreed upon by each synthetic expert in the group of multiple synthetic experts. The operations also include determining a final answer based on the respective group answer generated as output from the group of multiple synthetic experts. The final answer includes an adjudicated list of LoT for each of the number of different treatment regimens administered to the patient

This aspect of the disclosure may include one or more of the following optional features. In some implementations, each corresponding treatment regimen of the number of different treatment regiments administered to the patient is paired with a description of the corresponding treatment regimen, a start date indicating when the patient began the corresponding treatment regimen, and an end date indicating when the patient stopped the corresponding treatment regimen. In some additional implementations, the prompt composition further includes a format parameter that specifies how the LLM should format the respective group answer generated as output from the group of multiple synthetic experts. Here, the format of the respective group answer specified by the format parameter may include a list of elements containing numerical values of LoT for each treatment regimen of the number of different treatment regimens administered to the patient, wherein the format parameter specifies that a number of elements in the list of elements must be equal to the number of different treatment regimens.

In some examples, the prompt composition further includes one or more few-shot learning examples that each include exemplary natural language text characterizing example clinical data for an example patient being treated for the particular disease and a corresponding ground-truth list of LoT for each treatment regimen of the multiple different treatment regimens administered to the example patient. The example clinical data characterized by the natural language text includes multiple different treatment regimens administered to the example patient. In these examples, each few-shot learning example may further include a corresponding ground-truth chain-of-thought reasoning for why an expert would adjudicate the corresponding ground-truth list of LoT from the exemplary natural language text. Optionally, the instruction parameter that specifies the task for the LLM to synthesize the group of multiple synthetic experts that each use the adjudication rules to perform the chain-of-thought reasoning for making the LoT adjudication decisions may further specify that each synthetic expert is to also use the one or more few-shot learning examples to perform the chain-of-though reasoning for making the LoT adjudication decisions.

In some implementations, at least one of the different treatment regimens administered to the patient includes a combination of one or more drugs. Here, the prompt composition may further include a mechanism of action parameter that specifies: one or more drug classes commonly used to treat the particular disease; and for each corresponding drug class of the one or more drug classes, an example list of drugs within the corresponding drug class that have a same mechanism of action. Receiving the natural language input text may include receiving a clinical data table for the patient and serializing the clinical data table into the natural language input text. The clinical data table stores the clinical data for the patient in a tabular form.

In some examples, processing the adjudication prompt to cause the LLM to synthesize the group of synthetic experts includes, for each corresponding LLM instance of multiple LLM instances of the LLM, instructing the corresponding LLM instance to process the adjudication prompt independently from the other LLM instances to cause the corresponding LLM instance to synthesize a respective group of synthetic experts and generate a respective group answer as output from the respective group of multiple synthetic experts that indicates the predicted number of LoT agreed upon by each synthetic expert in the respective group of multiple synthetic experts. Here, the LLM may include a single pre-trained LLM executing each of the multiple LLM instances or the LLM may include two or more different pre-trained LLMs each executing one or more respective LLM instances among the multiple LLM instances. In these examples, the operations may further include determining a majority answer among the respective group answers generated as output from the respective groups of multiple synthetic experts synthesized by the multiple LLM instances of the LLM, wherein determining the final answer may include determining the final answer as the determined majority answer. Additionally, the operations may further include determining a majority vote percentage based on a total number of groups and a number of the respective groups of multiple synthetic experts that generated the same respective group answer as the majority answer.

The details of one or more implementations of the disclosure are set forth in the accompanying drawings and the description below. Other aspects, features, and advantages will be apparent from the description and drawings, and from the claims.

### DESCRIPTION OF DRAWINGS

FIG. 1 is a schematic view of a system for performing lines of therapy (LoT) adjudication automatically by leveraging a large language model (LLM).

FIG. 2A is a schematic view of an example tabular clinical data record.

FIG. 2B is a schematic view of example natural language input text characterizing clinical data.

FIG. 3A is a schematic view of example adjudication rules.

FIG. 3B is a schematic view of an example instruction parameter.

FIG. 3C is a schematic view of exemplary few-shot learning examples.

FIG. 3D is a schematic view of an example mechanism of action parameter.

FIGS. 4A-4C are example groups of synthetic group experts each performing chain-of-thought reasoning and collaborating with one another to reach agreement on a predicted number of LoT administered to a patient.

FIG. 5 is a flowchart of an example arrangement of operations for a method of performing LoT adjudication automatically a LLM.

FIG. 6 is a schematic view of an example computing device that may be used to implement the systems and methods described herein.

Like reference symbols in the various drawings indicate like elements.

### DETAILED DESCRIPTION

Clinical trial design (CTD) is a significant part of drug development as it impacts clinical trial length, protocol, patient enrollment, clinical endpoints, and comparator agents required. Knowledge from earlier clinical trials for a same drug, or a different drug, can help in the CTD process. The drug development process is a complex procedure where clinical trials are one of the lengthy and expensive components. Clinical trial documents include vast amounts of clinical information for different entities including disease entity, drug name, line of therapy, etc. Extracting and classifying these entities correctly may facilitate the design process of clinical trials. A line of therapy (LoT) is a series of ordered treatments given to patients during their disease progression. For example, a LoT for cancer treatment may include surgery first followed by chemotherapy and radiation. In this example, surgery is the first line of therapy, and chemotherapy and radiation are the second line of therapy. The LoT is an important concept used in many different clinical scenarios. For instance, in clinical trials, the LoT information of prior treatments can be used to include, or exclude, patients and then the current treatment may be placed as the next LoT. The LoT may also be reported in application materials for a regulatory approval of a drug. Moreover, doctors may choose a LoT for a patient based on his or her condition and provide treatment based on established guidelines pertinent to that LoT.

The LoT information may be used in different contexts such that the LoT information may be applied in several different applications. For instance, when designing a clinical trial of a drug, clinical trial designers may need to collect information from other clinical trials of similar drugs. In particular, the clinical trial designers may need to know which pivotal studies were used for the approval of a specific drug to leverage information from those pivotal studies. However, the regulatory approval documents may not explicitly mention the names and/or the National Clinical Trial (NCT) number of the corresponding clinical trial. This is known as the “trial matching problem,” where the clinical trial designer needs to find the clinical trials that were used for the approval of a specific drug. For instance, if a clinical trial designer wants to know which clinical trials were used for the regulatory approval of the drug “Accrufer” for the approval date of Jul. 25, 2019, then the answer may include the clinical trials “NCT01340872”, “NCT01352221”, and “NCT02968368”. To that end, different entities from the clinical trial documents may be used as well as the relationships between those entities that are common in the regulatory approval to identify the particular clinical trials. In some examples, the documents from the particular clinical trials used to approve a specific drug include entities such as disease entities, LoT, Treatment Regimen, name of drugs, co-treatments, a combination of drugs, bio-marker, disease sub-types, pathology, risk category, patient demographics, age cutoff, etc. Thus, correctly extracting and classifying these entities correctly may solve the trial matching problem. Moreover, different indications can be used for different approvals when a drug has multiple approvals. As such, the “unique indication identification problem” is when a clinical trial designer needs to know which indications were used for a particular approval. LoT information and other previously mentioned entities may similarly be extracted and classified to differentiate between multiple approvals of the same drug.

LoT is an important concept in the context of clinical trials as it may be relevant to the patient population for which a regulatory agency approves the drug. For example, regulatory agencies may grant approval for a first drug only to patients who have already failed the standard of care (SoC) therapy (e.g., first line of therapy), in which case the approval of the first drug would be referred to as second line of therapy approval. Similarly, if a second drug is only approved for patients for patients who have failed the SoC therapy and also therapy with the first drug, approval of the second drug would be referred to as a third line of therapy approval.

Accurate LoT categorization is vital for optimizing care, treatment strategies, medication assessments, clinical trial eligibility and clinical development. The assignment of LoT in processing Real-World Data (RWD) can be utilized to inform benchmark assumptions for clinical trial design, simultaneously facilitating the creation of a synthetic cohort that offers vital context for results from single-arm trials. However, challenges particularly in the RWD and Real-World Evidence (RWE) setting arise due to varied approaches, lack of standardized definitions, and clinician-dependent determination. Particularly within the context of RWD and RWE studies, the accuracy and timeliness of LoT assignment emerge as a pivotal initial step, subsequently affecting the allocation of index line/index date and the retrospective selection of subjects based on intended eligibility criteria. Manual assignment is time-consuming, resource-intensive, and subject to inter-observer/intra-observer variability. These challenges highlight the need for innovative solutions, such as the integration of artificial intelligence and machine learning (AI/ML). Table 1 below shows the number of clinicians required to manually adjudicate lines of therapy for patients across several studies.

TABLE 1
Metrics for manual adjudication.

| | # of pts | # of records | Time to | | # of |
|---|---|---|---|---|---|
| Study | adjudicated | adjudicated | adjudicate | | Clinicians |
| 1 | 1,290 | 11,464 | 3-4 | weeks | >5 |
| 2 | ~3,000 | 12,060 | 3 | weeks | 14 |
| 3 | ~1,800 | 4,157 | 2 | weeks | 4 |

Implementations herein are directed toward leveraging large language models (LLMs) of diversified knowledge to automatically adjudicate LoTs from clinical data with traceability. That is, LLMs that are pre-trained on large amounts of text may be leveraged to automatically adjudicate LoTs from clinical data. Specifically, implementations are directed toward leveraging LLMs to automatically adjudicate LoTs from extensive clinical data including treatment histories, disease progression, patient characteristics, and therapeutic outcomes. As will become apparent, techniques disclosed herein advantageously streamline the process, establishes standardized criteria, and enhances accuracy of adjudicating LoTs, while at the same time, allows clinical trial or treating physicians to focus on more nuanced patient care.

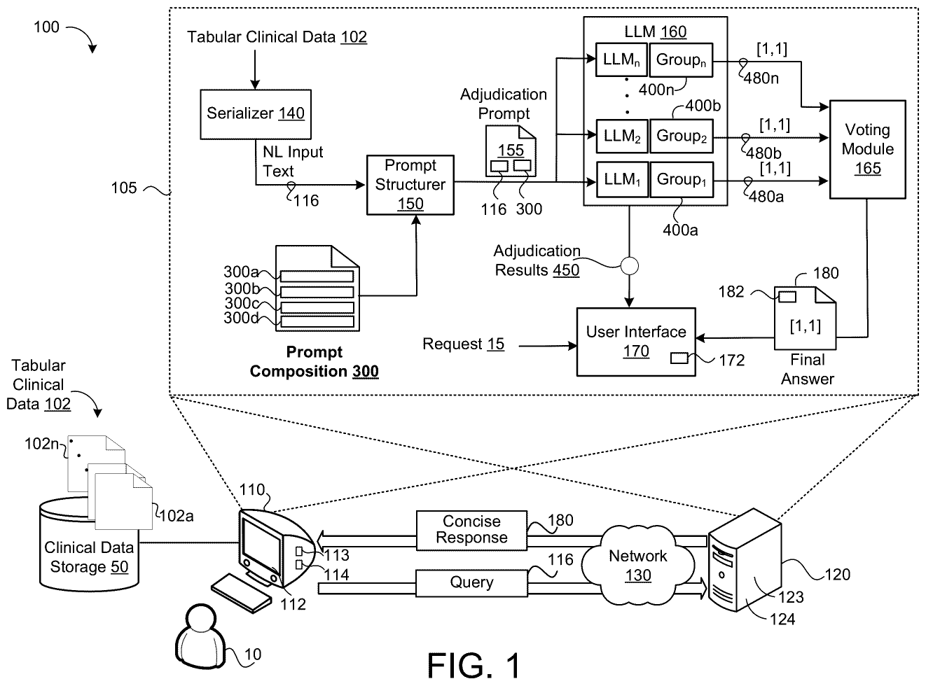

FIG. 1 illustrates an example system 100 for performing lines of therapy (LoT) adjudication automatically by leveraging a large language model (LLM) 160 to process natural language input text 116 characterizing clinical data 102 for a patient diagnosed with a particular disease. For instance, the particular disease may include cancer, and more specifically a specific type of cancer. Examples herein include Follicular Lymphoma as the particular disease, however, the techniques disclosed herein for performing LoT adjudication automatically are similarly applicable to other types of cancer or even diseases other than cancer where ascertaining LoT is required. An LoT application (or simply ‘application’) 105 may execute on a user device 110 associated with a user 10 (e.g., a healthcare provider or a clinical trial designer) to enable the user 10 and the LLM 160 to interact with one another. The application 105 may access various components for facilitating the interaction between the user 10 and the LLM 160 in a natural manner. For instance, through the use of application programming interfaces (APIs) or other types of plug-ins, the application 105 may access a clinical data serializer 140, a prompt structurer 150, the LLM 160, a voting module 165, and a user interface 170.

The system 100 includes the user device 110, a remote computing system 120, and a network 130. The user device 110 includes data processing hardware 113 and memory hardware 114. The user device 110 may be any computing device capable of communicating with the remote computing system 120 through the network 130. The user device 110 includes, but is not limited to, desktop computing devices and mobile computing devices, such as laptops, tablets, smart phones, smart speakers/displays, digital assistant devices, smart appliances, internet-of-things (IoT) devices, infotainment systems, vehicle infotainment systems, and wearable computing devices (e.g., headsets, smart glasses, and/or watches).

The remote computing system 120 may be a distributed system (e.g., a cloud computing environment) having scalable elastic resources. The resources include computing resources 123 (e.g., data processing hardware) and/or storage resources 124 (e.g., memory hardware). Additionally or alternatively, the remote computing system 120 may be a centralized system. The network 130 may be wired, wireless, or a combination thereof, and may include private networks and/or public networks, such as the Internet.

The components leveraged by the LoT application 105 may execute on the data processing hardware 113 of the user device 110 or on the data processing hardware 123 of the remote computing system 120. In some implementations, the components leveraged by the LoT application 105 executes on both the data processing hardware 113 of the user device 110 and the data processing hardware 123 of the remote computing system 120. For instance, one or more components of the LoT application 105 may execute on the data processing hardware 113 of the user device 110 while one or more other components of the LoT application 105 may execute on the remote computing system 120.

The LLM 160 may power the LoT assistant application 105 to provide personal chat bot capabilities for facilitating dialog conversations with the user 10 in natural language and performing tasks/actions on the user's behalf. In some examples, the LLM 160 includes an instance of Gemini, Bard, Meena, ChatGPT, or any other previously trained LLM. These previously trained LLMs have been previously trained on enormous amounts of diverse data and are capable of engaging in corresponding conversations with users in a natural and intuitive manner. However, these LLMs have a plurality of machine learning (ML) layers and hundreds of millions to hundreds of billions of ML parameters.

The user 10 uses the application 105 for leveraging the LLM 160 to adjudicate LoT from clinical data 102 for the patient diagnosed with the particular disease. The user device 110 may access clinical data storage 50 that stores tabular clinical data 102 for a number (n) of patients diagnosed with the particular disease. For instance, the clinical data storage 50 may store a respective tabular clinical data record 102a-n for each of the number of patients diagnosed with the particular disease. Here, a first tabular clinical data record 102a includes the clinical data for the first patient, a second tabular clinical data record 102b includes the clinical data for the second patient, and an n^{th} tabular clinical data record 102n includes the clinical data for the n^{th} patient.

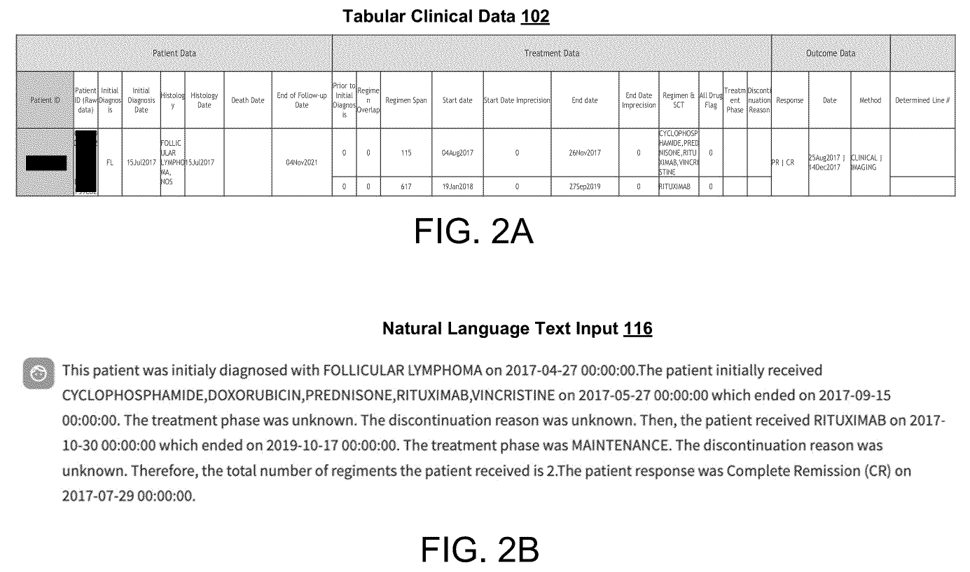

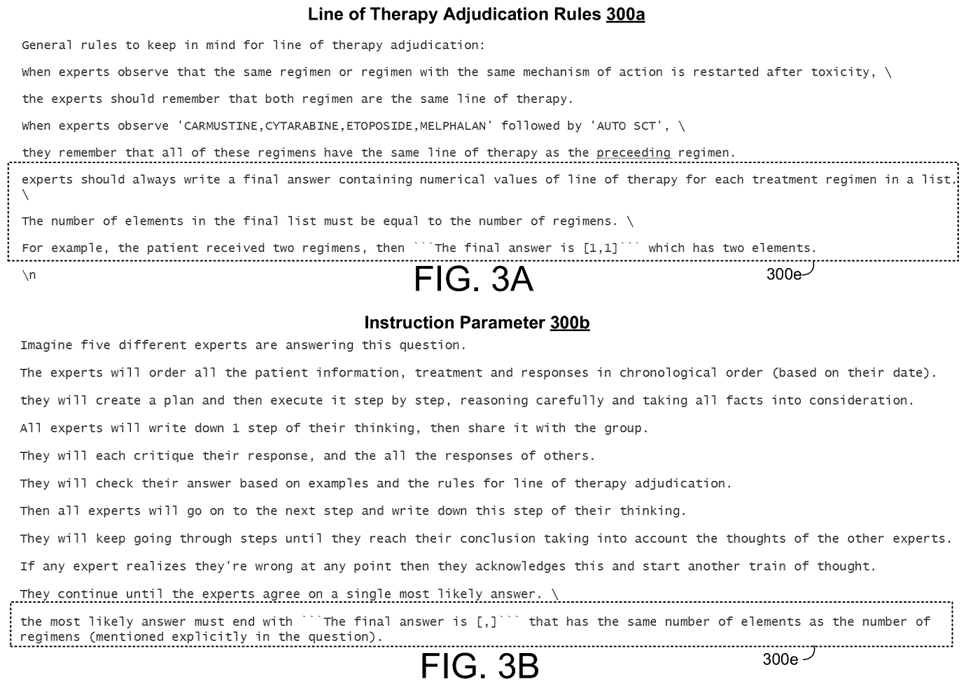

FIG. 2A provides an example of a tabular clinical data record 102 for a respective patient diagnosed with the particular disease. Here, the clinical data includes patient information, treatment information, and outcome information. The patient information includes a name for the particular disease (e.g., Follicular Lymphoma (FL)) the patient is diagnosed with and an initial diagnosis date of the particular disease. The patient information may also indicate whether or not histology imaging was performed, and if so, a date of the histology imaging. The patient information may also include a date of death of the patient died. If the patient did not die, the date of death may be left blank. If an end of follow-up for treating the particular disease of the patient occurred, the patient information in the tabular clinical data record 102 may include a date of the end of follow-up.

The treatment information included in the clinical data includes a number of different treatment regimens administered to the patient for treating the particular disease. In the example tabular clinical data record 102 shown in FIG. 2A, two different treatment regimens were administered to the patient. Each treatment regimen is further paired with a description of the corresponding treatment regimen, a start date indicating when the patient began the corresponding treatment regimen, and an end date indicating when the patient stopped the corresponding treatment regimen. The two treatment regimens administered to the patient in the example shown each include a respective combination of one or more drugs. For instance, the description of the first treatment regimen includes the drug combination of Cyclophosphamide, Prednisone, Rituximab, and Vincristine and the description of the second treatment regimen includes the drug Rituximab. Treatment regimens may include treatments other than medications such as, without limitation, chemotherapy, radiation, surgery, and more. For each treatment regimen, the treatment information may further indicate a treatment phase (e.g., maintenance, induction, conditioning, salvage, bridging, etc.) for when the treatment regimen was administered, a reason for discontinuation of the treatment regimen, a value for start date imprecision and end date imprecision indicating a level of imprecision for the date (e.g., 0=precise date; 1=at least 15 days; 2=at least 6 months; and 3=missing date), a span (in days) over which the treatment regimen was administered to the patient, as well as a value (in days) if the treatment regimen was administered prior to the date of the initial diagnosis of the particular disease. Reasons for discontinuation of a treatment regimen may include, without limitation, insurance reasons, disease progression, toxicity, inadequate response, death, doctor preference, patient preference, or unknown. In the example shown, a treatment phase of “maintenance” is labeled for when the treatment regimen of Rituximab was administered to the patient. The treatment information may further include a regimen overlap column indicating regimens with overlapping dates to provide a signal to the reviewer to assess outcomes in context with the overlapping treatment regimen as well as the current treatment regimen.

The outcome information included in the clinical data indicates one or more treatment responses obtained for each of the different treatment regimens administered to the patient. Each treatment response serves as an indicator of how the particular disease (e.g., cancer) has responded during or after a corresponding treatment regimen. For example, each treatment response may indicate a partial response or remission (PR), a complete response or remission (CR), stable disease (SD) indicating the particular disease has neither worsened or improved, and progression disease (PD) indicating that the particular disease has worsened (e.g., the cancer has grown or spread). The outcome information further includes a corresponding date for each treatment response indicating the date at which the treatment response was obtained. Treatment responses may be obtained for patients during follow-up visits with a physician (e.g., oncologist) who is treating the patient for the particular disease with the treatment regimen. These follow-up visits may continue for months or years after the treatment ends. Various tests/methodologies may be used to obtain an outcome result such as clinical testing or medical imaging. Clinical testing could include blood/urine/tissue tests measuring different substances in blood, like enzymes or proteins, that cancer cells or organs release when tumors grow. Medical imaging techniques may include, without limitation X-ray, computed tomography, magnetic resonance imaging, position emission tomography, or mammogram. As such, each treatment response in the outcome information may be further paired with a label indicating the technique used to obtain the corresponding treatment response. For instance, the label may include “Clinical” for clinical testing or “Imaging” for medical imaging.

The clinical data storage 50 may be stored on memory hardware 114 of the user device 110 or on memory hardware 114 of a remote computing device or server in communication with the user device 110. In some examples, the n patients diagnosed with the particular disease include patients that participated in clinical trials for approving drugs or other therapies for treating the particular disease. The user 10 may provide one or more tabular clinical data records 102a-n retrieved from the clinical data storage 50 to the application 105 for automatically performing LoT adjudication. For instance, the user device 110 may upload a batch of tabular clinical data records 102a-n and the application 105 may perform LoT adjudication on the clinical data for each patient represented by a respective tabular clinical data record. Similarly, the user device 110 may upload an individual tabular clinical data record 102 for the application 105 to automatically perform LoT adjudication. For simplicity, examples herein will describe a cycle of LoT adjudication performed on the clinical data for a single patient.

Notably, the LLM 160 may not be well suited for ingesting the tabular clinical data record 102 for a given patient. Here, the application 105 inputs the tabular clinical data record 102 into the serializer 140 for serializing the tabular clinical data record 102 into natural language (NL) input text 116. Here, the serializer 140 is configured to convert clinical data tables storing clinical data in a tabular form into natural language text inputs 116 so that the clinical data can be ingested/interpreted by the LLM 160. As such, the NL input text 116 includes natural language text describing the patient information, treatment information, and outcome information contained in the source tabular clinical data record 102. FIG. 2B shows example NL input text 116 characterizing clinical data for a patient diagnosed with the particular disease.

With continued reference to FIG. 1, the prompt structurer 150 receives the NL input text 116 and a prompt composition 300 and structurers an adjudication prompt 155 by concatenating the prompt composition 300 to the NL input text 116. As described in greater detail below, the application 105 issues the adjudication prompt 155 for input to the LLM 160 to cause one or more LLM instances (LLM₁, LLM₂, . . . LLMₙ) of the LLM 160 to each synthesize a respective group of multiple synthetic experts 400a-n (Group₁, Group₂, . . . Groupₙ) and generate a respective group answer 480a-n as output from the respective group of multiple synthetic experts. The group answer 480 indicates a predicted number of LoT agreed upon by each synthetic expert in the respective group of multiple synthetic experts. As used herein, the LLM 160 may include a single pre-trained LLM capable of executing all of the multiple LLM instances. Alternatively, the LLM 160 may include two or more different and uncorrelated pre-trained LLMs, each capable of executing one or more of the multiple LLM instances. As will become apparent, the adjudication prompt 155 can be input separately and independently to each of multiple LLM instances. Consequently, when the adjudication prompt 155 is input to multiple LLM instances each synthetizing a respective group of multiple synthetic experts 400 to generate a respective group answer 480, a single pre-trained LLM may execute all of the multiple LLM instances or two or more different pre-trained LLMs may each execute one or more respective LLM instances among the multiple LLM instances.

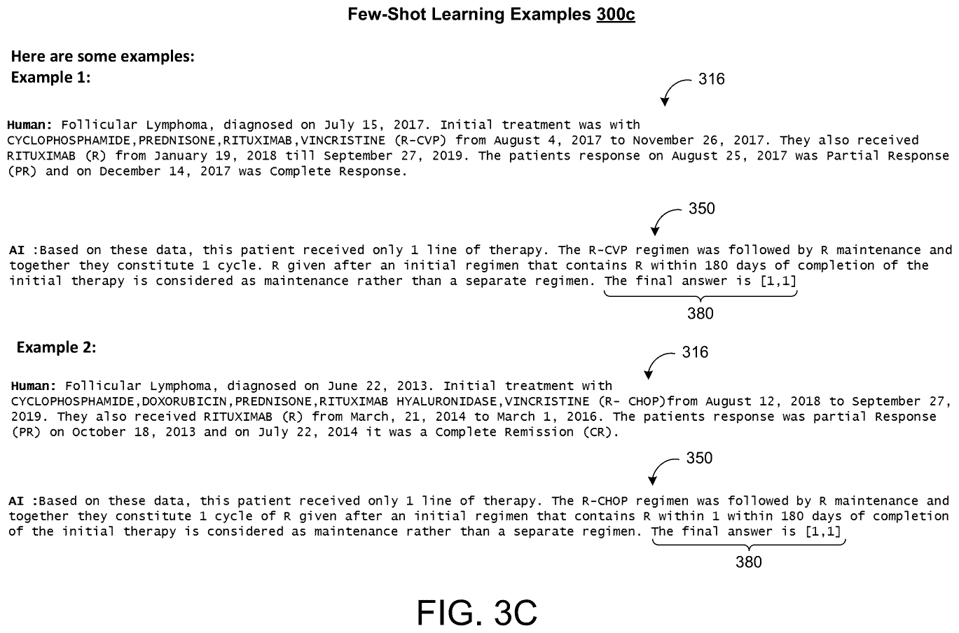

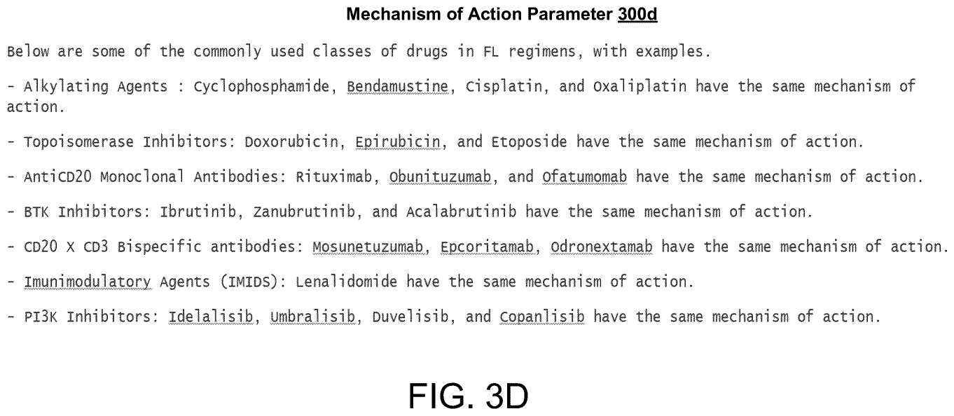

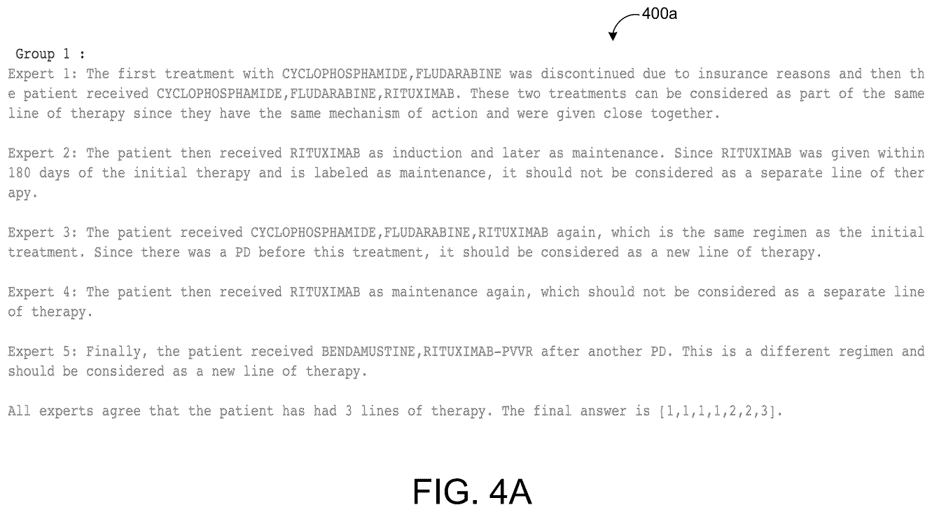

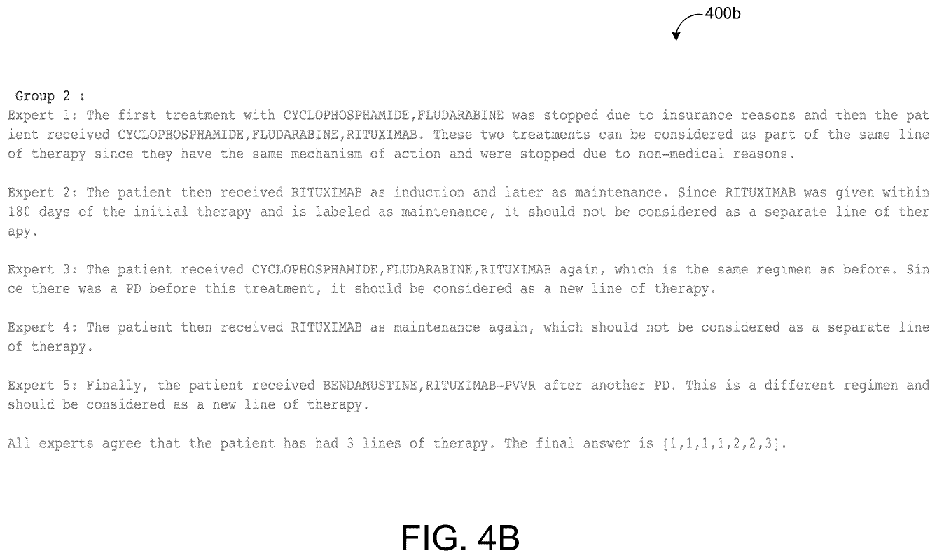

The prompt composition 300, when concatenated to the NL input text 116 to form the adjudication prompt 155, guides the LLM 160 to automatically perform LoT adjudication on the NL input text 116 without training or updating parameters of the pre-trained LLM 160. In the example shown, the prompt composition 300 includes adjudication rules 300a and instruction parameter 300b. The adjudication rules 300a specify rules for performing lines of therapy adjudication. FIG. 3A shows example adjudication rules 300a for performing LoT therapy adjudication on natural language text inputs 116 characterizing clinical data for patients diagnosed with the particular disease. The adjudication rules 300a are in natural language. In some examples, the adjudication rules 300a include a format parameter 300e of the prompt composition 300 that specifies how the LLM 160 should format a respective group answer 480 generated as output from each respective group of multiple synthetic experts 400. For instance, the format parameter 300e may specify that the format of a group answers 480 includes a list of elements containing numerical values for each treatment regimen of a number of different treatment regimens outlined in the clinical data characterized by the NL text input text 116. Here, the format parameter 300e may specify that a number of elements in the list of elements must be equal to the number of different treatment regimens. For instance, for a scenario with four different treatment regimens where the first two treatment regimens belong to a first LoT, the third treatment regimen belongs to a second LoT, and the fourth treatment regimen belongs to a third LoT, the final answer 480 would include the list of elements [1, 1, 2, 3].

The instruction parameter 300b specifies a task for the LLM 160 to synthesize the group of multiple synthetic experts that each use the adjudication rules 300a to perform chain-of-though reasoning for making LoT adjudication decisions based on the NL input text 116 and collaborate with one another to agree upon a predicted number of LoT administered to the patient. The predicted number of LoT agreed upon by each synthetic expert in the group of multiple synthetic experts 400 is conveyed in the respective group answer 480 output from the group of multiple synthetic experts 400. FIG. 3B shows an example instruction parameter 300b specifying the task for the LLM 160 to synthesize the group of multiple synthetic experts. The instruction parameter 300b may define the number of different synthetic experts that the LLM 160 should synthesize for performing the chain-of-thought reasoning for making the LoT adjudication decisions and for collaborating with the other synthetic experts in the group to agree upon the predicted number of LoT adjudicated from clinical data to convey in the respective group answer 480 output from the group. In the example shown in FIG. 3B, the instruction parameter 300b instructs the LLM 160 to synthesize five different synthetic experts. The instruction parameter 300b may specify any number of different synthetic experts to synthesize for the group. The number of different synthetic experts specified by the instruction parameter may be selected as a trade-off between latency and computational costs and accuracy of the group answer 480. For instance, more synthetic experts may provide more accurate predictions in the number of LoT adjudicated from clinical data, while at the same time, latency and computational costs increase.

The instruction parameter 300b instructs each synthetic expert to initially order all the patient information, treatment information, and outcome information of the clinical data in chronological order using the associated dates by processing the NL input text 116 characterizing the clinical data 102. Thereafter, each expert executes one step at a time by providing chain-of-thought reasoning for making an adjudication decision at the corresponding step by applying the adjudication rules 300a, and then sharing the chain-of-thought reasoning for the adjudication decision at the corresponding step with the other synthetic experts. As such, the synthetic experts may critique their adjudication decisions made at each step as well as the adjudication decisions made by the other synthetic experts. In some examples, when the prompt composition 300 provides one or more few-shot learning examples 300c, the instruction parameter 300b may further instruct each synthetic expert to also apply the few-shot learning examples 300c when performing the chain-of-thought reasoning for the adjudication decision at the corresponding step. The synthetic experts may continue to execute subsequent steps one at a time until the group of multiple synthetic experts agree upon the predicted number of LoT administered to the patient. In some examples, the instruction parameter 300b includes the format parameter 300e specifying how the respective group of multiple synthetic experts 400 should format the respective group answer 480 generated as output from the group.

Referring to FIGS. 1 and 3C, in some implementations, the prompt composition 300 additionally includes one or more few-shot learning examples 300c that provide in-context learning for enabling each synthetic expert synthesized by the pre-trained LLM 160 to generalize for the task of making LoT adjudication decisions. Each few-shot learning example 300c includes exemplary natural language text 316 characterizing example clinical data for an example patient being treated for the particular disease. The example clinical data characterized by the exemplary natural language text 316 including multiple different treatment regimens administered to the example patient. The natural language text 316 may further include corresponding dates for when the example patient was diagnosed with the particular disease and when each treatment regimen began and/or ended. The natural language text 316 may further include outcome information indicating clinical responses and dates thereof for each treatment regimen. Each few-shot learning example further includes a corresponding ground-truth list of LoT 380 paired with the exemplary natural language text 316. Here, the corresponding ground-truth list of LoT 380 indicates the list of LoT for each treatment regimen of the multiple different treatment regimens characterized by the exemplary natural language text 316. The ground-truth list of LoT 380 may include the same format as the format specified by the format parameter 300e for formatting the respective group answer 480 generated as output from the group of multiple synthetic experts 400. For instance, the example of FIG. 3C shows two few-shot learning examples each including exemplary natural language text 316 indicating that the corresponding example patient was administered two treatment regimens and a ground-truth list of LoT 380 for each of the two treatment regimens indicating that the two treatment regimens belong to a same LoT. That is, the ground-truth list of LoT 380 for each of the few-shot learning examples 300c (Example 1 and Example 2) is [1, 1]. The ground-truth list of LoT 380 may further prefix natural language text of “The final answer is”. In some examples, each few-shot learning example further includes a corresponding ground-truth chain-of-thought reasoning 350 for why an expert (e.g., synthetic expert) would adjudicate the corresponding ground-truth list of LoT 380 from the corresponding natural language text. The ground-truth chain-of-thought reasoning may apply the adjudication rules 300a of the composition prompt 300. The ground-truth chain-of-thought reasoning 350 may provide in-context learning for synthetic experts synthesized by the LLM 160 on how to generalize for performing chain-of-thought reasoning for the LoT adjudication decisions made at each step.

Referring back to FIG. 1, as previously discussed, a treatment regimen administered to the patient may include a combination of one or more drugs/medications. In some implementations, the prompt composition 300 further includes a mechanism of action parameter 300d. The mechanism of action parameter 300d may specify (in natural language text) one or more drug classes commonly used to treat the particular disease, and for each corresponding drug class of the one or more drug classes, an example list of drugs within the corresponding drug class that have a same mechanism of action. FIG. 3D shows an example mechanism of action parameter 300d specifying multiple different drug classes commonly used to treat Follicular Lymphoma (FL). Here, the different drug classes include Alkylating Agents, Topoisomerase Inhibitors, Anticd20 Monoclonal Antibodies, BTK Inhibitors, CD20×CD3 Bispecific Antibodies, Immunomodulatory Agents (IMIDS), and PI3K Inhibitors. For each corresponding drug class, the example mechanism of action parameter 300d lists multiple drugs within the corresponding drug class that have the same mechanism of action. Notably, the synthetic experts may use the mechanism of action parameter 300d included in the prompt composition 300 when making LoT adjudication decisions.

When performing LoT adjudication on multiple batches of clinical data for patients diagnosed with the particular disease, the prompt structurer 150 may structure a corresponding adjudication prompt 155 for each patient by concatenating the prompt composition 300 to corresponding natural language input text 116 characterizing the respective clinical data for each corresponding patient. After the prompt structurer 150 structures the adjudication prompt 155 by concatenating the prompt composition to the NL input text 116, the application 105 issues the adjudication prompt 155 for input to the LLM 160 to cause one or more LLM instances (LLM₁, LLM₂, . . . LLMₙ) of the LLM 160 (i.e., a single pre-trained LLM 160 or two or more different pre-trained LLMs 160) to each synthesize a respective group of multiple synthetic experts 400a-n (Group₁, Group₂, . . . Groupₙ) and generate a respective group answer 480a-n as output from the respective group of multiple synthetic experts. The group answer 480 indicates a predicted number of LoT agreed upon by each synthetic expert in the respective group of multiple synthetic experts. In some scenarios, the adjudication prompt 155 is issued as input to only a single LLM instance of the LLM 160 such that the LLM 160 processes the adjudication prompt 155 to generate a single group of multiple synthetic experts 400a and generate a single group answer 480a as output from the group of multiple synthetic experts 400a. In these scenarios, the group answer 480a generated as output includes a final answer 180 that the user interface 170 may provide as output from the user device 110 for the user. Here, the final answer 180 includes an adjudicated list of LoT for each of the number of different treatment regimens administered to the patient.

In the example shown, the adjudication prompt 155 is issued as input to multiple LLM instances (LLM₁, LLM₂, . . . LLMₙ) of the LLM 160 whereby each LLM instance processes the adjudication prompt 155 independently of the other LLM instances to result in each LLM instance of the multiple LLM instances synthesizing a respective group of synthetic experts 400a-n that ultimately generates a respective group answer 480a-n as output from the respective group of synthetic experts 400a-n. Specifically, for each corresponding LLM instance of the multiple LLM instances of the LLM 160, the application 105 may instruct the corresponding LLM instance to process the adjudication prompt 155 independently from the other LLM instances to cause the corresponding LLM instance to synthesize the respective group of synthetic experts 400 and generate the respective group answer 480 as output that indicates the predicted number of LoT agreed upon by each synthetic expert in the respective group of multiple synthetic experts 400. For example, when the instruction parameter 300b of the prompt composition 300 specifies the task for the LLMs of diversified knowledge 160 to synthesize a group of five (5) synthetic experts, each LLM instance of the multiple LLM instances (LLM₁, LLM₂, . . . LLMₙ) of the LLM 160 will synthesize a respective group of five (5) synthetic experts. For each respective group of synthetic experts 400 synthesized by the corresponding LLM instance, the instruction parameter 300b of the prompt composition 300 instructs each synthetic expert to initially order all the patient information, treatment information, and outcome information of the clinical data in chronological order using the associated dates by processing the NL input text 116 characterizing the clinical data 102. Thereafter, each expert executes one step at a time by providing chain-of-thought reasoning for making an adjudication decision at the corresponding step by applying the adjudication rules 300a (and optionally the few-shot learning examples 300c and/or mechanism of action parameter 300d when included in the prompt composition 300), and then sharing the chain-of-thought reasoning for the adjudication decision at the corresponding step with the other synthetic experts. As such, the synthetic experts may critique their adjudication decisions made at each step as well as the adjudication decisions made by the other synthetic experts. The synthetic experts may continue to execute subsequent steps one at a time until the group of multiple synthetic experts agree upon the predicted number of LoT administered to the patient. Each respective group of multiple synthetic experts 400a-n accordingly generates the respective group answer 480a-n as output that indicates the predicted number of LoT agreed upon by the synthetic experts. The LLM instances of the LLM 160 may format the respective group answers 480 according to the format specified by the format parameter 300e and also learned from the ground-truth list of LoT of the few-shot learning examples 300c.

With continued reference to FIG. 1, when the LLM 160 executes multiple LLM instances to synthesize multiple groups of synthetic experts each generating a single group answer 480a-n through collaborative critique among the synthetic experts with traceable chain-of-though reasoning, a voting module 165 receives the respective group answers 480a-n and determines a final answer 180 as a majority answer among the respective group answers 480a-n. The application 105 is configured to provide, for output from the user device 110, the final answer 180 determined by the voting module 165 as the majority answer among the group answers 480a-n generated as output from each group of multiple synthetic experts 400a-n. Here, the application 105 may instruct the user interface 170 to display, on a screen 112 in communication with the user device 110, the final answer 180. In some examples, the user interface 170 includes a text-to-speech (TTS) system that converts a textual representation of the final answer 180 into synthesized speech for audible output from the user device 110.

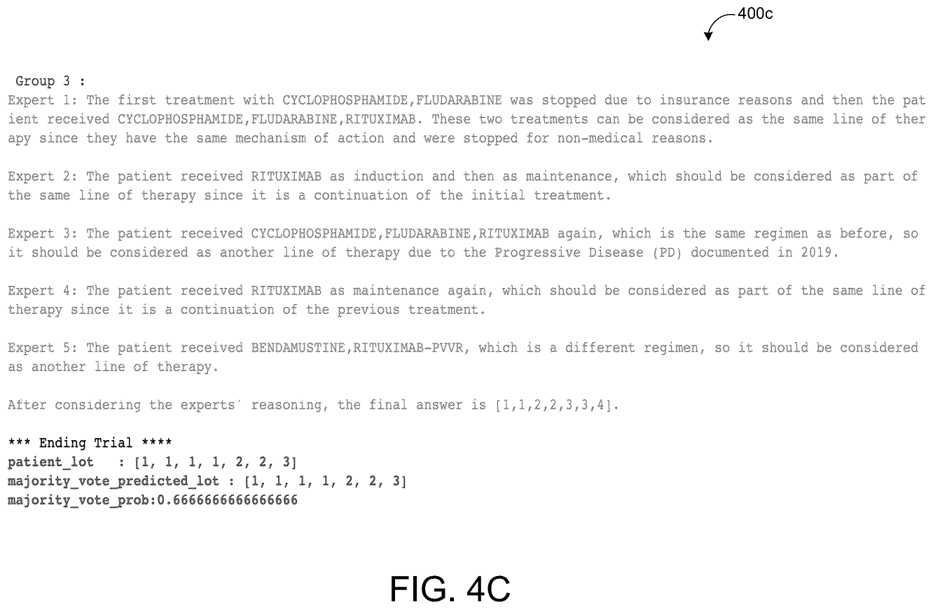

FIGS. 4A-4C show example groups of multiple synthetic experts 400a-c each synthesized by a respective LLM instance LLM₁, LLM₂, LLM₃ of the LLM 160 responsive to the LLM instances each receiving and processing the adjudication prompt 155 independently of the other LLM instances. FIG. 4A shows a first group of five synthetic experts 400a that each perform chain-of-thought reasoning for making LoT adjudication decisions based on the NL input text 116 and collaborate with one another to agree on a predicted number of LoT administered to the patient as a respective group answer 480a. For instance, the respective group answer 480a generated as output from the group may state, “All experts agree that the patient has had 3 lines of therapy. The final answer is [1, 1, 1, 1, 2, 2, 3]”. FIG. 4B shows a second group of five synthetic experts 400b that each perform chain-of-thought reasoning for making LoT adjudication decisions based on the NL input text 116 and collaborate with one another to agree on a predicted number of LoT administered to the patient as a respective group answer 480b. For instance, the respective group answer 480b generated as output from the group may state, “All experts agree that the patient has had 3 lines of therapy. The final answer is [1, 1, 1, 1, 2, 2, 3]”. Notably, the first and second groups of five synthetic experts 400a, 400b each generated the same group answers 480a, 480b. FIG. 4C shows a third group of five synthetic experts 400c that each perform chain-of-thought reasoning for making LoT adjudication decisions based on the NL input text 116 and collaborate with one another to agree on a predicted number of LoT administered to the patient as a respective group answer 480c. For instance, the respective group answer 480c generated as output from the group may state, “After considering the experts' reasoning, the final answer is [1, 1, 2, 2, 3, 3, 4]”. Here, group answer 480c indicates that the predicted number of LoT administered to the patient is equal to four (4), while the group answers 480a, 480b generated by the first and second groups predicts three (3) LoT administered to the patient. As such, the voting module 165 applies majority voting to determine the final answer 180 as the majority answer among the respective group answers. That is, the majority answer is three lines of therapy in the format [1, 1, 1, 1, 2, 2, 3].

The voting module 165 may also determine a majority vote percentage 182 based on a number of the respective groups of synthetic experts that generated the majority answer. The majority vote percentage 182 may serve as a confidence of the final answer 180 that conveys the list of LoT adjudicated from the clinical data 102 of the patient by the LLM 160. Notably, increasing the number of groups of synthetic experts may increase accuracy and confidence of the final answer 180 at the cost of increased latency and computational costs since the number of LLM instances executing increases proportionally to the number of groups of synthetic experts 400. As such, the number of groups of synthetic experts to synthesize may be selected to optimize LoT adjudication accuracy in view of latency and computational costs.

As shown in FIG. 1, the application 105 may append the majority vote percentage 182 to the final answer 180 provided as output from the user device 110 for the user 10. In some examples, the user interface 170 may output a notification from the user device 110 when the majority vote percentage fails to satisfy a confidence threshold. The notification may alert the user 10 that the final answer 180 determined from the clinical data for the corresponding patient so that the user 10 can investigate the reason for discrepancy between the group answers 480 generated as output from the different groups of multiple synthetic experts 400a-n. While FIG. 1 depicts the voting module 165 as a module/component separate from the LLM 160, the LLM 160 may be configured/adapted to perform the functionality of the voting module 165. For example, the LLM 160 may retain the final answers 180 independently generated by each LLM instance during a first roundtrip as historical context and then process the historical context to determine the majority answer during a second roundtrip.

The LLM 160 may output adjudication results 450 after processing the adjudication prompt 155 structured for the clinical data 102 for each patient diagnosed with the particular disease. The adjudication results 450 may be tagged with metadata or other identifier identifying the patient and/or a clinical trial for which the patient was a participant. As such, the user may issue requests 15 to the user interface 170 to retrieve adjudication results 450 of interest to the user 10. While not shown, the adjudication results 450 may be stored in the clinical data storage 50 or in another data storage location (e.g., data storage overlain on the memory hardware 114, 124). The adjudication results 450 may indicate the final answer 180 ascertained via majority voting, the majority vote percentage 182, the individual group answers 480 generated as output from the different respective groups of synthetic experts 400a-n independently synthesized by the LLM 160, and/or the chain-of-thought reasoning performed by each synthetic expert in each respective group of multiple synthetic experts. The adjudication results 450 may additionally include the natural language input text 116 and the prompt composition 300 that formed the corresponding adjudication prompt 155 issued to the LLM 160. For example, the adjudication results 450 may include all or any portion of the information conveyed by each group of multiple synthetic experts 400a-c of FIGS. 4A-4C. In some examples, the user request 15 may request adjudication results 450 for patients who were administered a particular drug during a specified LoT. For instance, the user request 15 may request adjudication results 450 for any patients that were administered Drug A as a second LoT. A user request 15 may further request adjudication results 450 for any patients that had negative treatment responses (e.g., progressive disease or death) following a prior positive treatment response (e.g., complete remission). The user request 15 may request any information conveyed by the adjudication prompt 155.

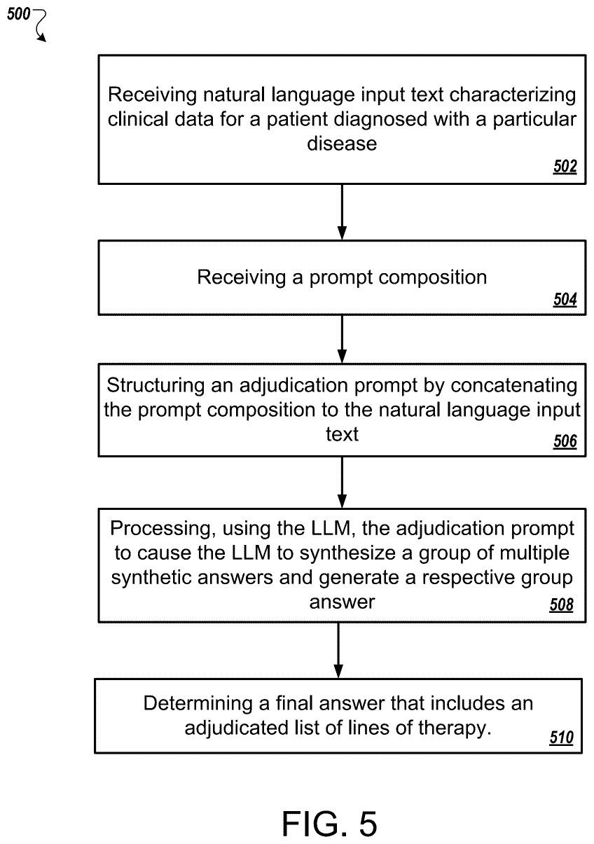

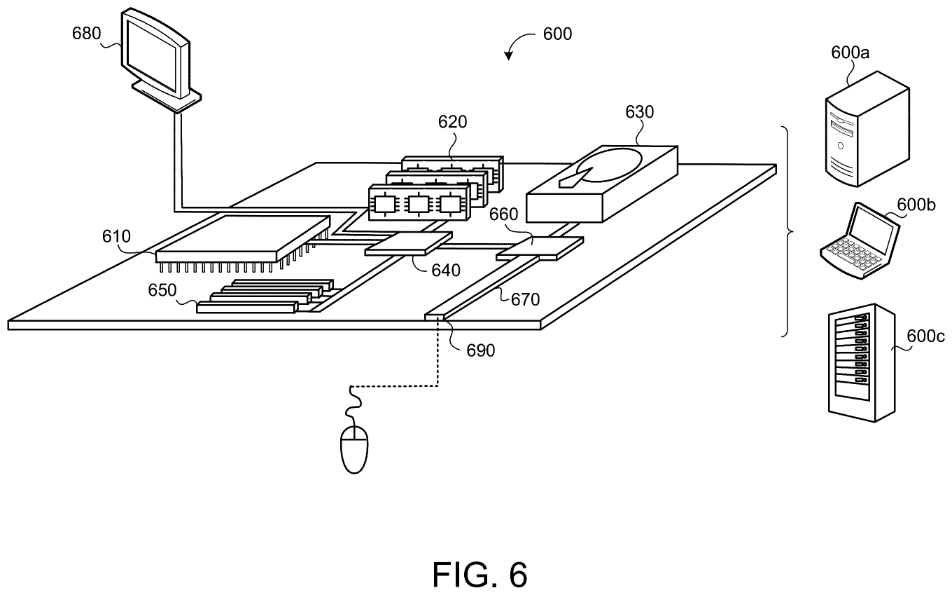

FIG. 5 is a flowchart of an example arrangement of operations for a method 500 of performing LoT adjudication automatically by leveraging a LLM 160 to process natural language input text 116 characterizing clinical data 102 for a patient diagnosed with a particular disease. The LLM 160 includes a pre-trained LLM 160. The method 500 may execute on data processing hardware 610 (FIG. 6) which may include the data processing hardware 113, 123 of the user device 110 and/or the remote computing device 120. At operation 502, the method 500 includes receiving the natural language input text 116 characterizing the clinical data 102 for the patient diagnosed with the particular disease. The clinical data includes patient information, treatment information, and outcome information. The patient information includes name of the particular disease the patient is diagnosed with and an initial diagnosis date of the particular disease. The treatment information includes a number of different treatment regimens administered to the patient for treating the particular disease. The outcome information indicates treatment responses to the treatment regimens administered to the patient.

At operation 504, the method 500 includes receiving a prompt composition 300 that includes adjudication rules 300a and an instruction parameter 300b. The adjudication rules 300a specify rules for performing LoT adjudication. The instruction parameter 300b specifies a task for the LLM 160 to synthesize a group of multiple synthetic experts 400 that each use the adjudication rules 300a to perform chain-of-thought reasoning for making LoT adjudication decisions based on the natural language input text 116 and collaborate with one another to agree upon a predicted number of LoT administered to the patient. The prompt composition 300 may also include one or more-few shot learning examples 300c to provide in-context learning for enabling each synthetic expert synthesized by the pre-trained LLM 160 to generalize for the task of making LoT adjudications. The prompt composition 300 may also include a mechanism of action parameter 300d. The prompt composition 300 may additionally include a format parameter 300e that specifies how the LLM should format the respective group answer generated as output from the group of multiple synthetic experts.

At operation 506, the method 500 includes structuring an adjudication prompt 155 by concatenating the prompt composition 300 to the natural language input text 116. At operation 508, the method 500 includes processing, using the LLM 160, the adjudication prompt 155 to cause the LLM 160 to synthesize the group of multiple synthetic experts 400 and generate a respective group answer 480 as output from the group of multiple synthetic experts 400. Here, the respective group answer 480 includes a predicted number of LoT agreed upon by each synthetic expert in the group of multiple synthetic answers. The respective group answer 480 generated by the group may include a format specified by the format parameter 300e that includes a list of elements containing numerical values of LoT for each treatment regimen of the number of different treatment regimens administered to the patient. The format parameter 300e may specify that a number of elements in the list of elements must be equal to the number of different treatment regimens. In some examples, the adjudication prompt 155 is issued as input to each of multiple LLM instances of the LLM. Here, each corresponding LLM instance processes the adjudication prompt 155 independently from the other LLM instances to cause the corresponding LLM instance to synthesize a respective group of synthetic experts 400a-n and generate a respective group answer 480a-n as output from the respective group. Here, each respective group answer 480a-n indicates the predicted number of LoT agreed upon by each synthetic expert in the respective group of synthetic experts 400a-n

At operation 510, the method 500 includes determining a final answer 180 based on the respective group answer 480 generated as output from the group of multiple synthetic experts 400. The final answer 180 includes an adjudicated list of LoT for each of the number of different treatment regimens administered to the patient. In examples when the adjudication prompt 155 is issued as input to only a single LLM instance of the LLM to synthesize only a single group of multiple synthetic experts, the group answer 480 generated as output from the single group serves as the final answer 180. In other examples when multiple groups of multiple synthetic experts 400a-n are synthesized, determining the final answer 180 includes determining the final answer 180 as a majority answer among the respective group answers 480a-n generated as output from the respective groups of synthetic experts. Here, a voting module 165 may receive all the independently generated group answers 480a-n and determine the majority answer therefrom. The voting module 1645 may determine a majority vote percentage 182 based on a total number of the multiple groups of synthetic experts and a number of the groups of synthetic experts that generated the same respective group answer as the majority answer. The majority vote percentage 182 may be appended to the final answer 180. The method 500 may also include providing the final answer for output from a user device 110. For instance, the final answer 180 may be graphically displayed on a screen 112 in communication with the user device 110.

A software application (i.e., a software resource) may refer to computer software that causes a computing device to perform a task. In some examples, a software application may be referred to as an “application,” an “app,” or a “program.” Example applications include, but are not limited to, system diagnostic applications, system management applications, system maintenance applications, word processing applications, spreadsheet applications, messaging applications, media streaming applications, social networking applications, and gaming applications.

The non-transitory memory may be physical devices used to store programs (e.g., sequences of instructions) or data (e.g., program state information) on a temporary or permanent basis for use by a computing device. The non-transitory memory may be volatile and/or non-volatile addressable semiconductor memory. Examples of non-volatile memory include, but are not limited to, flash memory and read-only memory (ROM)/programmable read-only memory (PROM)/erasable programmable read-only memory (EPROM)/electronically erasable programmable read-only memory (EEPROM) (e.g., typically used for firmware, such as boot programs). Examples of volatile memory include, but are not limited to, random access memory (RAM), dynamic random access memory (DRAM), static random access memory (SRAM), phase change memory (PCM) as well as disks or tapes.

FIG. 6 is schematic view of an example computing device 600 that may be used to implement the systems and methods described in this document. The computing device 600 is intended to represent various forms of digital computers, such as laptops, desktops, workstations, personal digital assistants, servers, blade servers, mainframes, and other appropriate computers. The components shown here, their connections and relationships, and their functions, are meant to be exemplary only, and are not meant to limit implementations of the inventions described and/or claimed in this document.

The computing device 600 includes a processor 610, memory 620, a storage device 630, a high-speed interface/controller 640 connecting to the memory 620 and high-speed expansion ports 650, and a low speed interface/controller 660 connecting to a low speed bus 670 and a storage device 630. Each of the components 610, 620, 630, 640, 650, and 660, are interconnected using various busses, and may be mounted on a common motherboard or in other manners as appropriate. The processor 610 can process instructions for execution within the computing device 600, including instructions stored in the memory 620 or on the storage device 630 to display graphical information for a graphical user interface (GUI) on an external input/output device, such as display 680 coupled to high speed interface 640. In other implementations, multiple processors and/or multiple buses may be used, as appropriate, along with multiple memories and types of memory. Also, multiple computing devices 600 may be connected, with each device providing portions of the necessary operations (e.g., as a server bank, a group of blade servers, or a multi-processor system).

The memory 620 stores information non-transitorily within the computing device 600. The memory 620 may be a computer-readable medium, a volatile memory unit(s), or non-volatile memory unit(s). The non-transitory memory 620 may be physical devices used to store programs (e.g., sequences of instructions) or data (e.g., program state information) on a temporary or permanent basis for use by the computing device 600. Examples of non-volatile memory include, but are not limited to, flash memory and read-only memory (ROM)/programmable read-only memory (PROM)/erasable programmable read-only memory (EPROM)/electronically erasable programmable read-only memory (EEPROM) (e.g., typically used for firmware, such as boot programs). Examples of volatile memory include, but are not limited to, random access memory (RAM), dynamic random access memory (DRAM), static random access memory (SRAM), phase change memory (PCM) as well as disks or tapes.

The storage device 630 is capable of providing mass storage for the computing device 600. In some implementations, the storage device 630 is a computer-readable medium. In various different implementations, the storage device 630 may be a floppy disk device, a hard disk device, an optical disk device, or a tape device, a flash memory or other similar solid state memory device, or an array of devices, including devices in a storage area network or other configurations. In additional implementations, a computer program product is tangibly embodied in an information carrier. The computer program product contains instructions that, when executed, perform one or more methods, such as those described above. The information carrier is a computer- or machine-readable medium, such as the memory 620, the storage device 630, or memory on processor 610.

The high speed controller 640 manages bandwidth-intensive operations for the computing device 600, while the low speed controller 660 manages lower bandwidth-intensive operations. Such allocation of duties is exemplary only. In some implementations, the high-speed controller 640 is coupled to the memory 620, the display 680 (e.g., through a graphics processor or accelerator), and to the high-speed expansion ports 650, which may accept various expansion cards (not shown). In some implementations, the low-speed controller 660 is coupled to the storage device 630 and a low-speed expansion port 690. The low-speed expansion port 690, which may include various communication ports (e.g., USB, Bluetooth, Ethernet, wireless Ethernet), may be coupled to one or more input/output devices, such as a keyboard, a pointing device, a scanner, or a networking device such as a switch or router, e.g., through a network adapter.

The computing device 600 may be implemented in a number of different forms, as shown in the figure. For example, it may be implemented as a standard server 600a or multiple times in a group of such servers 600a, as a laptop computer 600b, or as part of a rack server system 600c.

Various implementations of the systems and techniques described herein can be realized in digital electronic and/or optical circuitry, integrated circuitry, specially designed ASICs (application specific integrated circuits), computer hardware, firmware, software, and/or combinations thereof. These various implementations can include implementation in one or more computer programs that are executable and/or interpretable on a programmable system including at least one programmable processor, which may be special or general purpose, coupled to receive data and instructions from, and to transmit data and instructions to, a storage system, at least one input device, and at least one output device.

These computer programs (also known as programs, software, software applications or code) include machine instructions for a programmable processor, and can be implemented in a high-level procedural and/or object-oriented programming language, and/or in assembly/machine language. As used herein, the terms “machine-readable medium” and “computer-readable medium” refer to any computer program product, non-transitory computer readable medium, apparatus and/or device (e.g., magnetic discs, optical disks, memory, Programmable Logic Devices (PLDs)) used to provide machine instructions and/or data to a programmable processor, including a machine-readable medium that receives machine instructions as a machine-readable signal. The term “machine-readable signal” refers to any signal used to provide machine instructions and/or data to a programmable processor.

The processes and logic flows described in this specification can be performed by one or more programmable processors, also referred to as data processing hardware, executing one or more computer programs to perform functions by operating on input data and generating output. The processes and logic flows can also be performed by special purpose logic circuitry, e.g., an FPGA (field programmable gate array) or an ASIC (application specific integrated circuit). Processors suitable for the execution of a computer program include, by way of example, both general and special purpose microprocessors, and any one or more processors of any kind of digital computer. Generally, a processor will receive instructions and data from a read only memory or a random access memory or both. The essential elements of a computer are a processor for performing instructions and one or more memory devices for storing instructions and data. Generally, a computer will also include, or be operatively coupled to receive data from or transfer data to, or both, one or more mass storage devices for storing data, e.g., magnetic, magneto optical disks, or optical disks. However, a computer need not have such devices. Computer readable media suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto optical disks; and CD ROM and DVD-ROM disks. The processor and the memory can be supplemented by, or incorporated in, special purpose logic circuitry.

To provide for interaction with a user, one or more aspects of the disclosure can be implemented on a computer having a display device, e.g., a CRT (cathode ray tube), LCD (liquid crystal display) monitor, or touch screen for displaying information to the user and optionally a keyboard and a pointing device, e.g., a mouse or a trackball, by which the user can provide input to the computer. Other kinds of devices can be used to provide interaction with a user as well; for example, feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user; for example, by sending web pages to a web browser on a user's client device in response to requests received from the web browser.

A number of implementations have been described. Nevertheless, it will be understood that various modifications may be made without departing from the spirit and scope of the disclosure. Accordingly, other implementations are within the scope of the following claims.

What is claimed is:

1. A computer-implemented method executed on data processing hardware that causes the data processing hardware to perform operations comprising:
receiving natural language input text characterizing clinical data for a patient diagnosed with a particular disease, the clinical data comprising:
patient information comprising a name of the particular disease the patient is diagnosed with and an initial diagnosis date of the particular disease;
treatment information comprising a number of different treatment regimens administered to the patient for treating the particular disease; and
outcome information indicating treatment responses to the treatment regimens administered to the patient;
receiving a prompt composition comprising:
adjudication rules that specify rules for performing lines of therapy adjudication; and
an instruction parameter that specifies a task for a large language model (LLM) to synthesize a group of multiple synthetic experts that each use the adjudication rules to perform chain-of-thought reasoning for making lines of therapy (LoT) adjudication decisions based on the natural language input text and collaborate with one another to agree upon a predicted number of LoT administered to the patient;
structuring an adjudication prompt by concatenating the prompt composition to the natural language input text;
processing, using the LLM, the adjudication prompt to cause the LLM to synthesize the group of multiple synthetic experts and generate a respective group answer as output from the group of multiple synthetic experts, the group answer indicating the predicted number of LoT agreed upon by each synthetic expert in the group of multiple synthetic experts; and
determining a final answer based on the respective group answer generated as output from the group of multiple synthetic experts, the final answer comprising an adjudicated list of LoT for each of the number of different treatment regimens administered to the patient.

2. The computer-implemented method of claim 1, wherein each corresponding treatment regimen of the number of different treatment regiments administered to the patient is paired with a description of the corresponding treatment regimen, a start date indicating when the patient began the corresponding treatment regimen, and an end date indicating when the patient stopped the corresponding treatment regimen.

3. The computer-implemented method of claim 1, wherein the prompt composition further comprises a format parameter that specifies how the LLM should format the respective group answer generated as output from the group of multiple synthetic experts.

4. The computer-implemented method of claim 3, wherein the format of the respective group answer specified by the format parameter comprises a list of elements containing numerical values of LoT for each treatment regimen of the number of different treatment regimens administered to the patient, wherein the format parameter specifies that a number of elements in the list of elements must be equal to the number of different treatment regimens.

5. The computer-implemented method of claim 1, wherein the prompt composition further comprises one or more few-shot learning examples, each few-shot learning example comprising:
exemplary natural language text characterizing example clinical data for an example patient being treated for the particular disease, the example clinical data characterized by the natural language text comprising multiple different treatment regimens administered to the example patient; and
a corresponding ground-truth list of LoT for each treatment regimen of the multiple different treatment regimens administered to the example patient.

6. The computer-implemented method of claim 5, wherein each few-shot learning example further comprises a corresponding ground-truth chain-of-thought reasoning for why an expert would adjudicate the corresponding ground-truth list of LoT from the exemplary natural language text.

7. The computer-implemented method of claim 5, wherein the instruction parameter that specifies the task for the LLM to synthesize the group of multiple synthetic experts that each use the adjudication rules to perform the chain-of-thought reasoning for making the LoT adjudication decisions further specifies that each synthetic expert is to also use the one or more few-shot learning examples to perform the chain-of-though reasoning for making the LoT adjudication decisions.

8. The computer-implemented method of claim 1, wherein at least one of the different treatment regimens administered to the patient comprises a combination of one or more drugs.

9. The computer-implemented method of claim 8, wherein the prompt composition further comprises a mechanism of action parameter that specifies:
one or more drug classes commonly used to treat the particular disease; and
for each corresponding drug class of the one or more drug classes, an example list of drugs within the corresponding drug class that have a same mechanism of action.

10. The computer-implemented method of claim 1, wherein processing the adjudication prompt to cause the LLM to synthesize the group of synthetic experts comprises, for each corresponding LLM instance of multiple LLM instances of the LLM, instructing the corresponding LLM instance to process the adjudication prompt independently from the other LLM instances to cause the corresponding LLM instance to synthesize a respective group of synthetic experts and generate a respective group answer as output from the respective group of multiple synthetic experts that indicates the predicted number of LoT agreed upon by each synthetic expert in the respective group of multiple synthetic experts.

11. The computer-implemented method of claim 10, wherein the LLM comprises a single pre-trained LLM executing each of the multiple LLM instances.

12. The computer-implemented method of claim 10, wherein the LLM comprises two or more different pre-trained LLMs each executing one or more respective LLM instances among the multiple LLM instances.

13. The computer-implemented method of claim 10, wherein the operations further comprise:
determining a majority answer among the respective group answers generated as output from the respective groups of multiple synthetic experts synthesized by the multiple LLM instances of the LLM,
wherein determining the final answer comprises determining the final answer as the determined majority answer.

14. The computer-implemented method of claim 13, wherein the operations further comprise determining a majority vote percentage based on a total number of groups and a number of the respective groups of multiple synthetic experts that generated the same respective group answer as the majority answer.

15. The computer-implemented method of claim 1, wherein receiving the natural language input text comprises:
receiving a clinical data table for the patient, the clinical data table storing the clinical data for the patient in a tabular form; and
serializing the clinical data table into the natural language input text.

16. A system comprising:
data processing hardware; and
memory hardware in communication with the data processing hardware, the memory hardware storing instructions that when executed on the data processing hardware cause the data processing hardware to perform operations comprising:
receiving natural language input text characterizing clinical data for a patient diagnosed with a particular disease, the clinical data comprising:
patient information comprising a name of the particular disease the patient is diagnosed with and an initial diagnosis date of the particular disease;
treatment information comprising a number of different treatment regimens administered to the patient for treating the particular disease; and
outcome information indicating treatment responses to the treatment regimens administered to the patient;
receiving a prompt composition comprising:
adjudication rules that specify rules for performing lines of therapy adjudication; and
an instruction parameter that specifies a task for a large language model (LLM) to synthesize a group of multiple synthetic experts that each use the adjudication rules to perform chain-of-thought reasoning for making lines of therapy (LoT) adjudication decisions based on the natural language input text and collaborate with one another to agree upon a predicted number of LoT administered to the patient;
structuring an adjudication prompt by concatenating the prompt composition to the natural language input text;
processing, using the LLM, the adjudication prompt to cause the LLM to synthesize the group of multiple synthetic experts and generate a respective group answer as output from the group of multiple synthetic experts, the group answer indicating the predicted number of LoT agreed upon by each synthetic expert in the group of multiple synthetic experts; and
determining a final answer based on the respective group answer generated as output from the group of multiple synthetic experts, the final answer comprising an adjudicated list of LoT for each of the number of different treatment regimens administered to the patient.

17. The system of claim 16, wherein each corresponding treatment regimen of the number of different treatment regiments administered to the patient is paired with a description of the corresponding treatment regimen, a start date indicating when the patient began the corresponding treatment regimen, and an end date indicating when the patient stopped the corresponding treatment regimen.

18. The system of claim 16, wherein the prompt composition further comprises a format parameter that specifies how the LLM should format the respective group answer generated as output from the group of multiple synthetic experts.

19. The system of claim 18, wherein the format of the respective group answer specified by the format parameter comprises a list of elements containing numerical values of LoT for each treatment regimen of the number of different treatment regimens administered to the patient, wherein the format parameter specifies that a number of elements in the list of elements must be equal to the number of different treatment regimens.

20. The system of claim 16, wherein the prompt composition further comprises one or more few-shot learning examples, each few-shot learning example comprising:
exemplary natural language text characterizing example clinical data for an example patient being treated for the particular disease, the example clinical data characterized by the natural language text comprising multiple different treatment regimens administered to the example patient; and
a corresponding ground-truth list of LoT for each treatment regimen of the multiple different treatment regimens administered to the example patient.

21. The system of claim 20, wherein each few-shot learning example further comprises a corresponding ground-truth chain-of-thought reasoning for why an expert would adjudicate the corresponding ground-truth list of LoT from the exemplary natural language text.

22. The system of claim 20, wherein the instruction parameter that specifies the task for the LLM to synthesize the group of multiple synthetic experts that each use the adjudication rules to perform the chain-of-thought reasoning for making the LoT adjudication decisions further specifies that each synthetic expert is to also use the one or more few-shot learning examples to perform the chain-of-though reasoning for making the LoT adjudication decisions.

23. The system of claim 16, wherein at least one of the different treatment regimens administered to the patient comprises a combination of one or more drugs.

24. The system of claim 23, wherein the prompt composition further comprises a mechanism of action parameter that specifies:
one or more drug classes commonly used to treat the particular disease; and
for each corresponding drug class of the one or more drug classes, an example list of drugs within the corresponding drug class that have a same mechanism of action.

25. The system of claim 16, wherein processing the adjudication prompt to cause the LLM to synthesize the group of synthetic experts comprises, for each corresponding LLM instance of multiple LLM instances of the LLM, instructing the corresponding LLM instance to process the adjudication prompt independently from the other LLM instances to cause the corresponding LLM instance to synthesize a respective group of synthetic experts and generate a respective group answer as output from the respective group of multiple synthetic experts that indicates the predicted number of LoT agreed upon by each synthetic expert in the respective group of multiple synthetic experts.

26. The system of claim 25, wherein the LLM comprises a single pre-trained LLM executing each of the multiple LLM instances.

27. The system of claim 25, wherein the LLM comprises two or more different pre-trained LLMs each executing one or more respective LLM instances among the multiple LLM instances.

28. The system of claim 25, wherein the operations further comprise:
determining a majority answer among the respective group answers generated as output from the respective groups of multiple synthetic experts synthesized by the multiple LLM instances of the LLM,
wherein determining the final answer comprises determining the final answer as the determined majority answer.

29. The system of claim 28, wherein the operations further comprise determining a majority vote percentage based on a total number of groups and a number of the respective groups of multiple synthetic experts that generated the same respective group answer as the majority answer.

30. The system of claim 16, wherein receiving the natural language input text comprises:
receiving a clinical data table for the patient, the clinical data table storing the clinical data for the patient in a tabular form; and
serializing the clinical data table into the natural language input text.
